(19) United States
(12) Patent Application Publication
(10) Pub. No.: US 2025/0366524 A1
(43) Pub. Date: Dec. 4, 2025

(54) HEAT-NOT-BURN (HNB) AEROSOL-GENERATING DEVICES CONFIGURED FOR SPLIT OR BYPASS FLOW, CAPSULES FOR SUCH DEVICES, AND METHODS OF GENERATING AN AEROSOL

(71) Applicant: Altria Client Services LLC, Richmond, VA (US)

(72) Inventors: Ali A. ROSTAMI, Glen Allen, VA (US); Nicolas D. CASTRO, Richmond, VA (US)

(73) Assignee: Altria Client Services LLC, Richmond, VA (US)

(21) Appl. No.: 19/176,258

(22) Filed: Apr. 11, 2025

Related U.S. Application Data
(60) Provisional application No. 63/634,964, filed on Apr. 17, 2024.

(51) Int. Cl. A24F 40/485; A24F 40/20; A24F 40/42; A24F 40/46; A61M 15/06; H05B 3/02

(52) U.S. Cl. CPC A24F 40/485; A24F 40/20; A24F 40/42; A24F 40/46; H05B 3/02; A61M 15/06

(57) ABSTRACT

An aerosol-generating device may include an aerosol-forming substrate and a device body configured to receive the aerosol-forming substrate and an incoming airflow. The device body is additionally configured to direct a target airflow of the incoming airflow through the aerosol-forming substrate while the aerosol-forming substrate is heated such that a heated outflow exits therefrom. The device body is further configured to combine the heated outflow with a secondary airflow to produce a mixed flow. The secondary airflow is an air stream that has not passed through the aerosol-forming substrate. The aerosol-forming substrate may be contained in a capsule that is configured to split the incoming airflow into the target airflow and the secondary airflow.

### CROSS-REFERENCE TO RELATED APPLICATION

This application claims priority under 35 U.S.C. § 119 to U.S. Provisional Application No. 63/634,964, filed on Apr. 17, 2024, the entire contents of which are hereby incorporated herein by reference.

### BACKGROUND

### Field

The present disclosure relates to heat-not-burn (HNB) aerosol-generating devices, capsules for such devices, and methods of generating an aerosol without involving a substantial pyrolysis of the aerosol-forming substrate.

### Description of Related Art

Some electronic devices are configured to heat a plant material to a temperature that is sufficient to release constituents of the plant material while keeping the temperature below its ignition temperature so as to avoid a self-sustaining burning or a self-sustaining combustion of the plant material (i.e., in contrast to where a plant material is lit, such as lit-end cigarettes). Such devices may be characterized as generating an aerosol of constituents released by heating, and may be referred to as heat-not-burn aerosol-generating devices, or heat-not-burn devices.

### SUMMARY

At least one embodiment relates to a heat-not-burn (HNB) aerosol-generating device. In an example embodiment, the aerosol-generating device may include an aerosol-forming substrate and a device body configured to receive the aerosol-forming substrate and an incoming airflow. The device body may be additionally configured to direct a target airflow of the incoming airflow through the aerosol-forming substrate while the aerosol-forming substrate is heated such that a heated outflow exits therefrom. The device body may be further configured to combine the heated outflow with a secondary airflow to produce a mixed flow. The secondary airflow is an air stream that has not passed through the aerosol-forming substrate.

At least one embodiment relates to a capsule for a heat-not-burn (HNB) aerosol-generating device. In an example embodiment, the capsule may include a housing defining a substrate chamber, a chamber inlet, a chamber outlet, a bypass channel, a channel inlet, and a channel outlet. The capsule may additionally include an aerosol-forming substrate within the substrate chamber of the housing. The housing may be configured to split an incoming airflow into a target airflow and a secondary airflow such that the target airflow is directed through the aerosol-forming substrate via the chamber inlet, the substrate chamber, and the chamber outlet and such that the secondary airflow bypasses the aerosol-forming substrate via the channel inlet, the bypass channel, and the channel outlet.

At least one embodiment relates to a method of generating an aerosol. In an example embodiment, the method may include heating an aerosol-forming substrate and combining a heated outflow from the aerosol-forming substrate with a secondary airflow to produce a mixed flow. The secondary airflow is an air stream that has not passed through the aerosol-forming substrate.

### BRIEF DESCRIPTION OF THE DRAWINGS

The various features and advantages of the non-limiting embodiments herein may become more apparent upon review of the detailed description in conjunction with the accompanying drawings. The accompanying drawings are merely provided for illustrative purposes and should not be interpreted to limit the scope of the claims. The accompanying drawings are not to be considered as drawn to scale unless explicitly noted. For purposes of clarity, various dimensions of the drawings may have been exaggerated.

FIG. 1 is a schematic view of an aerosol-generating device according to an example embodiment.

FIG. 2 is a cross-sectional view of another aerosol-generating device according to an example embodiment.

FIG. 3 is a plan view of a flow distributor according to an example embodiment.

FIG. 4 is a plan view of another flow distributor according to an example embodiment.

FIG. 5 is a cross-sectional view of another aerosol-generating device according to an example embodiment.

FIG. 6 is a plan view of another flow distributor according to an example embodiment.

FIG. 7 is a cross-sectional view of another aerosol-generating device according to an example embodiment.

FIG. 8 is a cross-sectional view of another aerosol-generating device according to an example embodiment.

FIG. 9 is a perspective view of another aerosol-generating device according to an example embodiment.

FIG. 10 is a perspective view of another aerosol-generating device according to an example embodiment.

FIG. 11 is a downstream, perspective view of the aerosol-forming article in FIG. 10.

FIG. 12 is an upstream, perspective view of the aerosol-forming article in FIG. 10.

FIG. 13 is a perspective view of the cover of the aerosol-forming article in FIG. 10.

FIG. 14 is a perspective view of the second end cap of the aerosol-forming article in FIG. 10.

FIG. 15 is a perspective view of the first end cap of the aerosol-forming article in FIG. 10.

FIG. 16 is a thermal imaging view of an aerosol-forming article wherein all of the incoming airflow is passing through the aerosol-forming substrate.

FIG. 17 is a thermal imaging view of an aerosol-forming article wherein only a fraction of the incoming airflow is passing through the aerosol-forming substrate.

### DETAILED DESCRIPTION

Some detailed example embodiments are disclosed herein. However, specific structural and functional details disclosed herein are merely representative for purposes of describing example embodiments. Example embodiments may, however, be embodied in many alternate forms and should not be construed as limited to only the example embodiments set forth herein.

Accordingly, while example embodiments are capable of various modifications and alternative forms, example embodiments thereof are shown by way of example in the drawings and will herein be described in detail. It should be understood, however, that there is no intent to limit example embodiments to the particular forms disclosed, but to the contrary, example embodiments are to cover all modifications, equivalents, and alternatives thereof. Like numbers refer to like elements throughout the description of the figures.

It should be understood that when an element or layer is referred to as being “on,” “connected to,” “coupled to,” “attached to,” “adjacent to,” or “covering” another element or layer, it may be directly on, connected to, coupled to, attached to, adjacent to or covering the other element or layer or intervening elements or layers may be present. In contrast, when an element is referred to as being “directly on,” “directly connected to,” or “directly coupled to” another element or layer, there are no intervening elements or layers present. Like numbers refer to like elements throughout the specification. As used herein, the term “and/or” includes any and all combinations or sub-combinations of one or more of the associated listed items.

It should be understood that, although the terms first, second, third, etc. may be used herein to describe various elements, regions, layers and/or sections, these elements, regions, layers, and/or sections should not be limited by these terms. These terms are only used to distinguish one element, region, layer, or section from another region, layer, or section. Thus, a first element, region, layer, or section discussed below could be termed a second element, region, layer, or section without departing from the teachings of example embodiments.

Spatially relative terms (e.g., “beneath,” “below,” “lower,” “above,” “upper,” and the like) may be used herein for ease of description to describe one element or feature's relationship to another element(s) or feature(s) as illustrated in the figures. It should be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” may encompass both an orientation of above and below. The device may be otherwise oriented (rotated 90 degrees or at other orientations) and the spatially relative descriptors used herein interpreted accordingly.

The terminology used herein is for the purpose of describing various example embodiments only and is not intended to be limiting of example embodiments. As used herein, the singular forms “a,” “an,” and “the” are intended to include the plural forms as well, unless the context clearly indicates otherwise. It will be further understood that the terms “includes,” “including,” “comprises,” and/or “comprising,” when used in this specification, specify the presence of stated features, integers, steps, operations, and/or elements, but do not preclude the presence or addition of one or more other features, integers, steps, operations, elements, and/or groups thereof.

When the terms “about” or “substantially” are used in this specification in connection with a numerical value, it is intended that the associated numerical value includes a manufacturing or operational tolerance (e.g., ±10%) around the stated numerical value. Moreover, when the terms “generally” or “substantially” are used in connection with geometric shapes, it is intended that precision of the geometric shape is not required but that latitude for the shape is within the scope of the disclosure. Furthermore, regardless of whether numerical values or shapes are modified as “about,” “generally,” or “substantially,” it will be understood that these values and shapes should be construed as including a manufacturing or operational tolerance (e.g., ±10%) around the stated numerical values or shapes.

Unless otherwise defined, all terms (including technical and scientific terms) used herein have the same meaning as commonly understood by one of ordinary skill in the art to which example embodiments belong. It will be further understood that terms, including those defined in commonly used dictionaries, should be interpreted as having a meaning that is consistent with their meaning in the context of the relevant art and will not be interpreted in an idealized or overly formal sense unless expressly so defined herein.

The processing circuitry may be hardware including logic circuits; a hardware/software combination such as a processor executing software; or a combination thereof. For example, the processing circuitry more specifically may include, but is not limited to, a central processing unit (CPU), an arithmetic logic unit (ALU), a digital signal processor, a microcomputer, a field programmable gate array (FPGA), a System-on-Chip (SoC), a programmable logic unit, a microprocessor, application-specific integrated circuit (ASIC), etc.

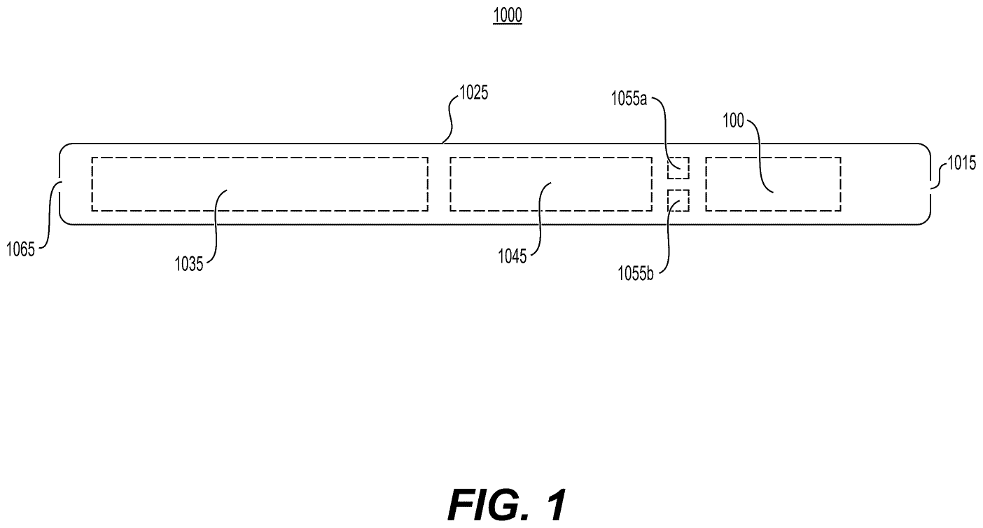

FIG. 1 is a schematic view of an aerosol-generating device according to an example embodiment. As shown in FIG. 1, the aerosol-generating device 1000 includes an aerosol-forming article 100 (e.g., aerosol-forming substrate, capsule including the same, etc.). A device body 1025 is configured to receive the aerosol-forming article 100, and an incoming airflow. For example, the incoming airflow may enter the device body 1025 via an air inlet 1065, which may include one or more openings for allowing airflow into the device body 1025. Although the air inlet 1065 is schematically shown as being defined by the upstream end of the device body 1025, it should be understood that the air inlet 1065 may additionally or alternatively defined by the upstream sidewall and/or the downstream sidewall of the device body 1025.

The device body 1025 is configured to direct a target airflow of the incoming airflow through the aerosol-forming article 100 while the aerosol-forming article 100 is heated, such that a heated outflow exits the aerosol-forming article 100. The device body 1025 is configured to combine the heated outflow with a secondary airflow to produce a mixed flow. The secondary airflow may be an air stream that has not passed through the aerosol-forming article 100. For example, the incoming airflow in the device body 1025 may be split, so that only a portion (e.g., fraction) of puff air flows through the aerosol-forming article 100. The remaining portion of the split airflow (e.g., bypass air) may flow through the device body 1025 without passing through the aerosol-forming article 100, and then mix with a heated outflow exiting the aerosol-forming article 100, to provide rapid cooling and aerosol formation. Alternatively, the secondary airflow may be ambient air that was not split from the incoming airflow but rather is one or more independent flows drawn into the device body 1025 via one or more separate inlets for combination with the heated outflow to produce a mixed flow.

In some example embodiments, a heat-not-burn tobacco device (such as the aerosol-generating device 1000) may heat at least a portion of tobacco filler (and/or other suitable aerosol-forming substrate in an aerosol-forming article) to above 150 degrees Celsius (or higher or lower temperatures). As a result, some tobacco constituents are released due to volatilization and are entrained with the air flowing therethrough. In contrast to devices where all puff air passes through the tobacco filler, some example embodiments may include a device body 1025 that splits the same puff air into two or more parts and/or draws independent flows of ambient air into the device body 1025 via one or more separate inlets.

For example, some of the puff air (e.g., an incoming airflow via the air inlet 1065) may pass through the heated aerosol-forming article 100 (which may be referred to as a target airflow, filler air, a heated outflow, etc.), and the remaining air may bypass the aerosol-forming article 100 (which may be referred to as bypass air, secondary airflow, etc.). The bypass air may be mixed with the heated outflow downstream of the aerosol-forming article 100.

The aerosol-forming article 100 may be heated transiently by an electric resistive heater as the target airflow passes through the aerosol-forming article 100, where the target airflow carries volatiles generated by heating the aerosol-forming article 100. For resistive heating, the resistive heater may be embedded within the aerosol-forming article 100 (for inside-out heating) and/or arranged externally to the aerosol-forming article 100 (for outside-in heating). Alternatively, the aerosol-forming article 100 may be inductively heated via internal and/or external susceptors. Once the volatile-containing heated air exits the aerosol-forming article 100, it mixes with the cooler bypass air resulting in rapid cooling of the mixture. This cooling may result in a change of the mixture to a supersaturated state, and may trigger nucleation and condensation aerosol.

The filler air carries the volatiles out of the aerosol-forming article 100, but may also cool the aerosol-forming article 100, contrary to an intended purpose of heating the aerosol-forming article 100. In an energy efficient system, it may be desirable to have most of the energy consumed for heating the aerosol-forming article 100, and any heat transferred to the flowing filler air may be considered as a waste for energy if it does not contribute to aerosol formation. The amount of each constituent released during this heating process depends on the temperature of the aerosol-forming article 100, the duration of heating, and the amount of corresponding precursors in the starting aerosol-forming article 100.

In some example embodiments, thermal and airflow analysis were performed using computational fluid dynamics, to compare an arrangement where all of the inlet air flows through the aerosol-forming article 100, and an arrangement where the inlet air is split so only a portion goes through the aerosol-forming article 100. The temperature of the aerosol-forming article 100 in the split flow configuration is higher, and the distribution is more uniform, than the configuration where all of the inlet air flows through the aerosol-forming article 100.

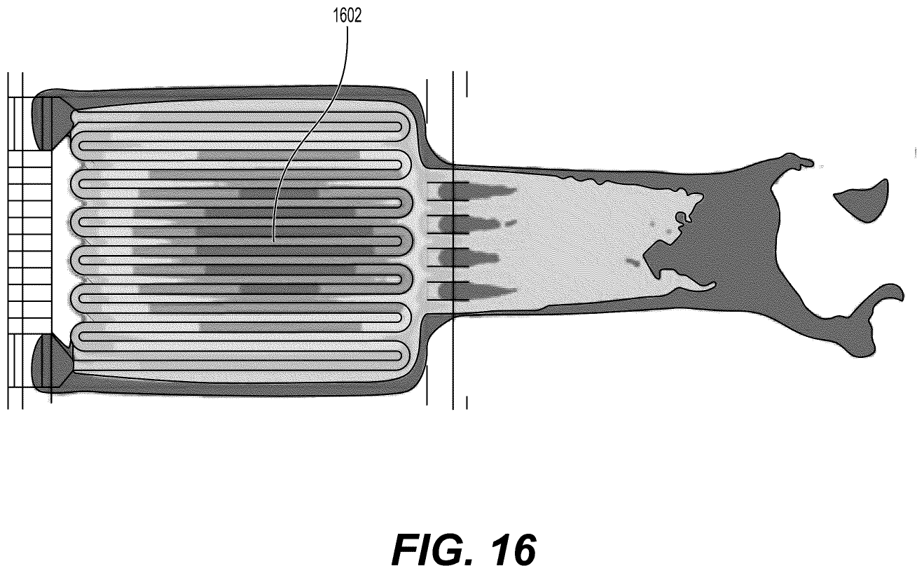

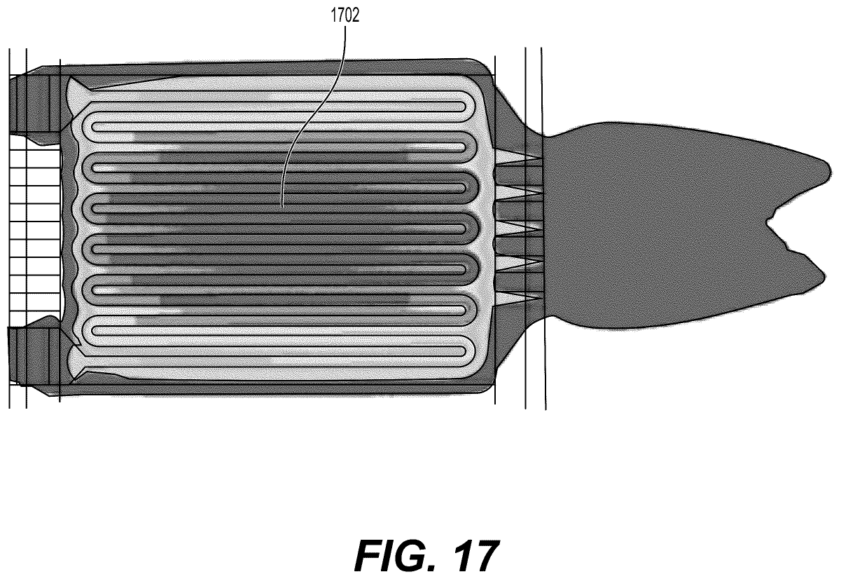

Example thermal captures of the split flow configuration versus the all inlet air configuration are illustrated in FIGS. 16 and 17 and discussed below. The snapshots were created at the end of a two second puff. Thermal efficiency of the system may be measured based on a fraction of total filler mass heated above a specified temperature. In some example embodiments, when 100% of the inlet air flows through the aerosol-forming article 100, 69.2% of filler mass reaches above 150 degrees Celsius. This number increases to 86.5% when 10% of air flows through filler and 90% bypasses the filler, as shown in Table 1 discussed below with reference to FIGS. 16 and 17.

In some example embodiments, a flow distributor mechanism may be used to split an airflow into a specific ratio. For example, one or more baffles may be placed upstream and/or downstream of the aerosol-forming article 100, with a specified number and size of holes to divide the inlet airflow into a target airflow and a secondary airflow. Example baffle configurations are described herein for splitting the airflow into two or more different flows.

For example, a flow distributor having three holes may allow a target airflow through a center hole for flowing through the aerosol-forming article 100, and side holes that allow bypass air to flow around the aerosol-forming article 100 without physically interacting with it. Airflow may be split in any desired range between greater than 0% and less than 100% of the air flowing through the aerosol-forming article 100, by adjusting a size and of the holes (e.g., orifices, etc.).

Some example embodiments may provide one or more of the following advantages: higher temperatures of the aerosol-forming article 100, more uniform temperature distribution in the aerosol-forming article 100, increased utilization of the aerosol-forming article 100 and increased aerosol mass, increased control of the resistance to draw (RTD), increased energy efficiency and battery life, lower aerosol temperature, and smaller particle sizes.

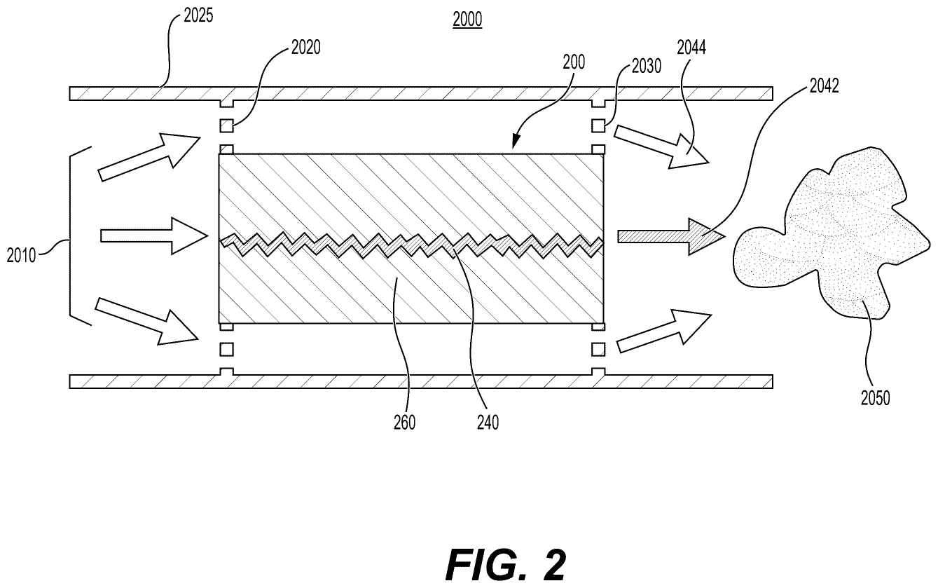

FIG. 2 is a cross-sectional view of another aerosol-generating device 2000 according to an example embodiment. As shown in FIG. 2, the aerosol-generating device 2000 includes a device body 2025, which should be understood to be a schematic depiction of just one of various possible configurations. The device body 2025 is configured to receive an incoming airflow 2010, which may be received at an end (e.g., upstream end) of the device body 2025. The device body 2025 is configured to split the incoming airflow 2010. For example, the device body 2025 includes a flow distributor 2020, which may define one or more holes for splitting the incoming airflow 2010.

The aerosol-generating device 2000 includes an aerosol-forming article 200. The aerosol-forming article 200 may include an aerosol-forming substrate 260, which may be heated by a heater 240. The heater 240 may be a planar heater which extends along the longitudinal axis of the aerosol-forming article 200. Alternatively, the heater 240 may be a corrugated heater which zigzags about the central plane/center line of the aerosol-forming article 200 as it extends along the longitudinal axis of the aerosol-forming article 200. Example embodiments of the heater 240 may be as disclosed in U.S. application Ser. No. 17/151,317, filed Jan. 18, 2021, titled “CLOSED SYSTEM CAPSULE WITH AIRFLOW, HEAT-NOT-BURN (HNB) AEROSOL-GENERATING DEVICES, AND METHODS OF GENERATING AN AEROSOL,” Atty. Dkt. No. 24000NV-000630-US; and U.S. application Ser. No. 17/137,468, filed Dec. 30, 2020, titled “CAPSULES INCLUDING EMBEDDED CORRUGATED HEATER, HEAT-NOT-BURN (HNB) AEROSOL-GENERATING DEVICES, AND METHODS OF GENERATING AN AEROSOL,” Atty. Dkt. No. 24000NV-000631-US, the disclosures of each of which are incorporated herein in their entirety by reference. Furthermore, as shown in FIG. 2, the flow distributor 2020 may split an incoming airflow 2010 into multiple air flows, such as a target airflow and a secondary airflow.

The target airflow may pass through the aerosol-forming substrate 260 of the aerosol-forming article 200, and may pass over, along, through, or otherwise in thermal communication with the heater 240. Therefore, the target airflow may entrain at least a portion of the volatiles released from the aerosol-forming substrate 260 as the aerosol-forming substrate 260 is heated by the heater 240. In some example embodiments, the flow distributor 2020 defines one or more holes for the target airflow that are aligned with the aerosol-forming substrate 260. For example, if the aerosol-forming substrate 260 is located in a central or axial portion of the device body 2025, the flow distributor 2020 may include an opening, hole, orifice, etc., for the target airflow that is located upstream of a central or axial portion of the device body 2025 where the aerosol-forming substrate 260 is located.

The flow distributor 2020 may additionally define one more holes that direct a secondary air flow around the aerosol-forming substrate 260. For example, the device body 2025 may include one or more bypass channels which go around a portion of the aerosol-forming substrate 260, such that the secondary airflow flowing through the bypass channel does not contact or flow through the aerosol-forming substrate 260. The flow distributor 2020 may include one or more holes aligned with one or more bypass channels (e.g., the holes of the flow distributor 2020 are upstream of the one or more bypass channels), such that the secondary airflow which is split by the flow distributor 2020 travels through the bypass channel and around the aerosol-forming substrate 260.

As shown in FIG. 2, the device body 2025 includes another flow distributor 2030 downstream of the aerosol-forming substrate 260. The flow distributor 2030 facilitates mixing of the secondary airflow 2044 (which did not pass through the aerosol-forming substrate 260), with a heated outflow 2042 exiting the aerosol-forming substrate 260. For example, the flow distributor 2030 includes one or more holes, orifices, openings, etc., aligned with the aerosol-forming article 200, such that air passing through the aerosol-forming substrate 260 and heated by the heater 240 may exit the flow distributor 2030 as the heated outflow 2042.

The flow distributor 2030 may include one or more openings aligned with one more bypass channels of the device body 2025, such that the secondary airflow 2044 exits the flow distributor 2030 to be mixed with the heated outflow 2042. The heated outflow 2042 and the secondary airflow 2044 may mix to provide an aerosol 2050.

In some example embodiments, a heat-not-burn (HNB) device (such as the aerosol-generating device 2000) is configured to split an incoming airflow 2010 such that only a fraction (e.g., a through flow) of the air flows through the aerosol-forming substrate 260 (e.g., tobacco) while a remainder (e.g., a bypass flow) of the incoming airflow 2010 flows around so as to bypass the aerosol-forming substrate 260. The incoming airflow 2010 may be split with the flow distributor 2020.

The aerosol-forming substrate 260 is heated with the internal heater 240, which may extend longitudinally within the aerosol-forming substrate 260 (or in any other suitable orientation), to release volatiles from the aerosol-forming substrate 260. The through flow of the air travels longitudinally through the aerosol-forming substrate 260 to entrain the released volatiles, while the bypass flow of the air moves in a concurrent manner but external to the aerosol-forming substrate 260 (e.g., through bypass channels). Upon exiting the aerosol-forming substrate 260, the heated outflow 2042 (and entrained volatiles) is mixed with the secondary airflow 2044 to generate an aerosol 2050 (e.g., condensation aerosol).

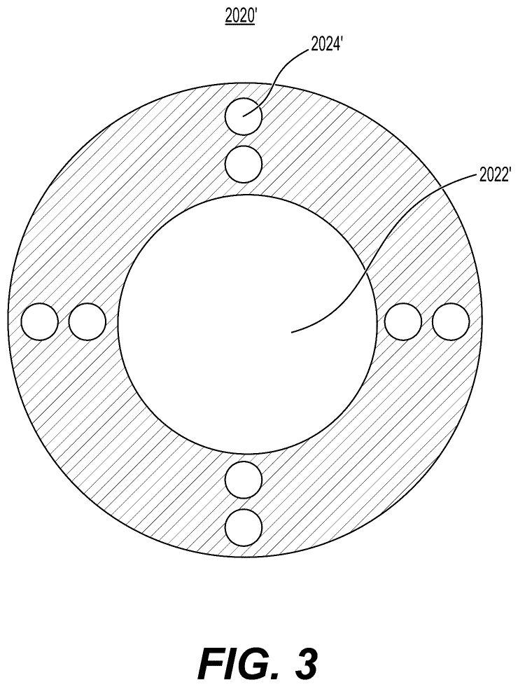

FIG. 3 is a plan view of a flow distributor 2020′ according to an example embodiment. As shown in FIG. 3, the flow distributor 2020′ includes a target opening 2022′, and multiple secondary openings 2024′. For example, the target opening 2022′ may allow a target airflow to pass through an aerosol-forming substrate (such as the aerosol-forming substrate 260 of FIG. 2). In some example embodiments, the target opening 2022′ may be aligned centrally, axially, or otherwise, with the aerosol-forming substrate, such that the target opening 2022′ is aligned upstream of the aerosol-forming substrate of the aerosol-generating device.

The secondary openings 2024′ may be aligned with one or more bypass channels, allowing secondary airflow to bypass the aerosol-forming substrate. For example, each secondary opening 2024′ may be aligned upstream of a bypass channel, such that a split air flow passing through the secondary opening 2024′ enters the bypass channel and does not flow through the aerosol-forming substrate.

Although FIG. 3 illustrates the flow distributor 2020′ as including one target opening 2022′ which is much larger than the eight smaller secondary openings 2024′, it should be understood that example embodiments are not limited thereto. In some example embodiments, the flow distributor 2020′ may include different quantities of the target opening 2022′ and secondary openings 2024′ (e.g., more target openings 2022′, more or less secondary openings 2024′), different sizes of secondary openings 2024′ and target openings 2022′, different positions of the target opening 2022′ and secondary openings 2024′, etc.

For example, the size, location, number, etc., of the target openings 2022′ and secondary openings 2024′ may correspond to locations of aerosol-forming substrates, bypass channels, etc., within the aerosol-generating device, such that the target opening 2022′ and the secondary openings 2024′ may or may not be aligned with corresponding aerosol-forming substrates, bypass channels, etc. In some example embodiments, the size and shape of the target openings 2022′ and secondary openings 2024′ may be adjusted to control a ratio of the flow of air through the target openings 2022′ and secondary openings 2024′ (such as by changing a diameter of the openings, etc.).

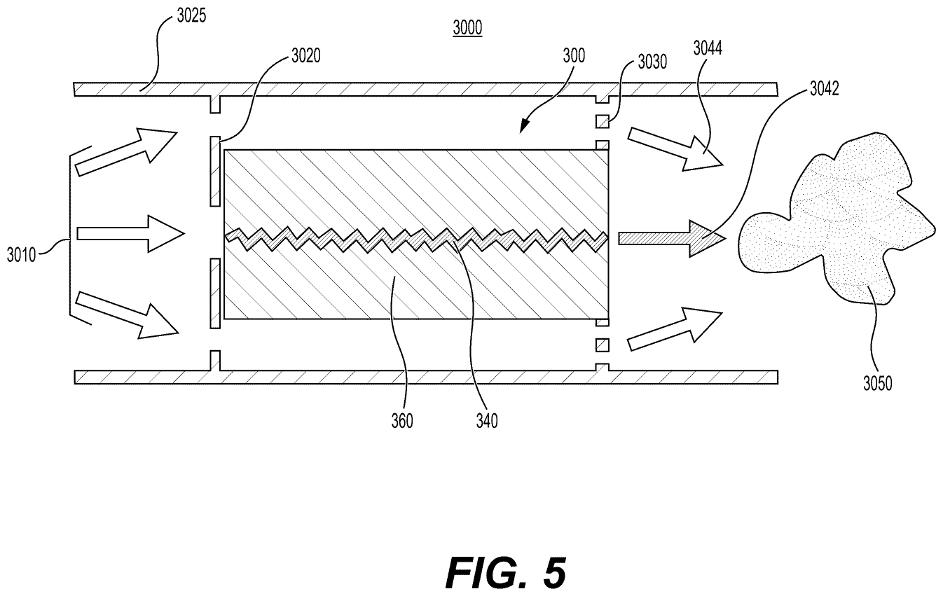

The flow distributor 2020′ may be in a form of a baffle that is disposed upstream of the aerosol-forming substrate. In an example embodiment, a flow distributor may define three holes (as illustrated in FIG. 5 discussed below), wherein the central hole provides the through flow while the two side holes provide the bypass flow. The holes can be sized depending on the properties of the aerosol-forming substrate and the desired split for the through flow and the bypass flow.

A majority of the incoming flow of air may be diverted as a bypass flow that flows around (e.g., rather than through) the aerosol-forming substrate. As a result, a higher and more uniform temperature may be achieved for the aerosol-forming substrate, which may yield more aerosol mass. In an example embodiment, at least 80% of the incoming flow of air is diverted as a bypass flow, while no more than 20% of the incoming flow of air is used as a through flow. In some example embodiments, at least 83% of the aerosol-forming substrate may reach a temperature above 150° C.

As discussed herein, an aerosol-forming substrate is a material or combination of materials that may yield an aerosol. An aerosol relates to the matter generated or output by the devices disclosed, claimed, and equivalents thereof. The material may include a compound (e.g., nicotine, cannabinoid), wherein an aerosol including the compound is produced when the material is heated.

It is understood that heating of a plant material below its ignition temperature may, in some circumstances, produce incidental and insubstantial levels of oxidized or other thermal decomposition byproducts. However, in some embodiments, the heating in aerosol-generating devices is below the pyrolysis temperature of the plant material so as to produce an aerosol having no or insubstantial levels of thermal decomposition byproducts of the plant material. Thus, in an example embodiment, pyrolysis of the plant material does not occur during the heating and resulting production of aerosol. In other instances, there may be incidental pyrolysis, with production of oxidized or other thermal decomposition byproducts at levels that are insignificant relative to the primary constituents released by heating of the plant materials.

The aerosol-forming substrate may be a fibrous material. For instance, the fibrous material may be a botanical material. The fibrous material is configured to release a compound when heated. The compound may be a naturally occurring constituent of the fibrous material. For instance, the fibrous material may be plant material such as tobacco, and the compound released may be nicotine. The term “tobacco” includes any tobacco plant material including tobacco leaf, tobacco plug, reconstituted tobacco, compressed tobacco, shaped tobacco, or powder tobacco, and combinations thereof from one or more species of tobacco plants, such as Nicotiana rustica and Nicotiana tabacum. In some example embodiments, the aerosol-forming substrate may include a plant material. The plant material may include tobacco.

In some example embodiments, the tobacco material may include material from any member of the genus Nicotiana. In addition, the tobacco material may include a blend of two or more different tobacco varieties. Examples of suitable types of tobacco materials that may be used include, but are not limited to, flue-cured tobacco, Burley tobacco, Dark tobacco, Maryland tobacco, Oriental tobacco, rare tobacco, specialty tobacco, blends thereof, and the like. The tobacco material may be provided in any suitable form, including, but not limited to, tobacco lamina, processed tobacco materials, such as volume expanded or puffed tobacco, processed tobacco stems, such as cut-rolled or cut-puffed stems, reconstituted tobacco materials, blends thereof, and the like. In some example embodiments, the tobacco material is in the form of a substantially dry tobacco mass. Furthermore, in some instances, the tobacco material may be mixed and/or combined with at least one of propylene glycol, glycerin, sub-combinations thereof, or combinations thereof.

The compound may also be a naturally occurring constituent of a medicinal plant that has a medically-accepted therapeutic effect. For instance, the medicinal plant may be a cannabis plant, and the compound may be a cannabinoid. Cannabinoids interact with receptors in the body to produce a wide range of effects. As a result, cannabinoids have been used for a variety of medicinal purposes (e.g., treatment of pain, nausea, epilepsy, psychiatric disorders). The fibrous material may include the leaf and/or flower material from one or more species of cannabis plants such as Cannabis sativa, Cannabis indica, and Cannabis ruderalis. In some instances, the fibrous material is a mixture of 60-80% (e.g., 70%) Cannabis sativa and 20-40% (e.g., 30%) Cannabis indica.

Examples of cannabinoids include tetrahydrocannabinolic acid (THCA), tetrahydrocannabinol (THC), cannabidiolic acid (CBDA), cannabidiol (CBD), cannabinol (CBN), cannabicyclol (CBL), cannabichromene (CBC), and cannabigerol (CBG). Tetrahydrocannabinolic acid (THCA) is a precursor of tetrahydrocannabinol (THC), while cannabidiolic acid (CBDA) is precursor of cannabidiol (CBD). Tetrahydrocannabinolic acid (THCA) and cannabidiolic acid (CBDA) may be converted to tetrahydrocannabinol (THC) and cannabidiol (CBD), respectively, via heating. In an example embodiment, heat from a heater (e.g., the heater 240 shown in FIG. 2) may cause decarboxylation so as to convert the tetrahydrocannabinolic acid (THCA) in the aerosol-forming article 200 to tetrahydrocannabinol (THC), and/or to convert the cannabidiolic acid (CBDA) in the aerosol-forming article 200 to cannabidiol (CBD).

In instances where both tetrahydrocannabinolic acid (THCA) and tetrahydrocannabinol (THC) are present in the aerosol-forming article 200, the decarboxylation and resulting conversion will cause a decrease in tetrahydrocannabinolic acid (THCA) and an increase in tetrahydrocannabinol (THC). At least 50% (e.g., at least 87%) of the tetrahydrocannabinolic acid (THCA) may be converted to tetrahydrocannabinol (THC) during the heating of the aerosol-forming article 200. Similarly, in instances where both cannabidiolic acid (CBDA) and cannabidiol (CBD) are present in the aerosol-forming article 200, the decarboxylation and resulting conversion will cause a decrease in cannabidiolic acid (CBDA) and an increase in cannabidiol (CBD). At least 50% (e.g., at least 87%) of the cannabidiolic acid (CBDA) may be converted to cannabidiol (CBD) during the heating of the aerosol-forming article 200.

Furthermore, the compound may be or may additionally include a non-naturally occurring additive that is subsequently introduced into the fibrous material. In one instance, the fibrous material may include at least one of cotton, polyethylene, polyester, rayon, combinations thereof, or the like (e.g., in a form of a gauze). In another instance, the fibrous material may be a cellulose material (e.g., non-tobacco and/or non-cannabis material). In either instance, the compound introduced may include nicotine, cannabinoids, and/or flavorants. The flavorants may be from natural sources, such as plant extracts (e.g., tobacco extract, cannabis extract), and/or artificial sources. In yet another instance, when the fibrous material includes tobacco and/or cannabis, the compound may be or may additionally include one or more flavorants (e.g., menthol, mint, vanilla). Thus, the compound within the aerosol-forming substrate may include naturally occurring constituents and/or non-naturally occurring additives. In this regard, it should be understood that existing levels of the naturally occurring constituents of the aerosol-forming substrate may be increased through supplementation. For example, the existing levels of nicotine in a quantity of tobacco may be increased through supplementation with an extract containing nicotine. Similarly, the existing levels of one or more cannabinoids in a quantity of cannabis may be increased through supplementation with an extract containing such cannabinoids.

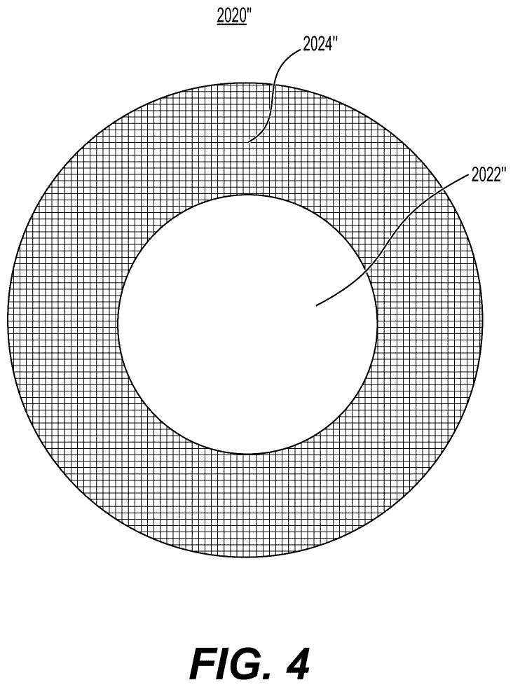

FIG. 4 is a plan view of another flow distributor 2020″ according to an example embodiment. As shown in FIG. 4, the flow distributor 2020″ includes a target opening 2022″ and a plurality of secondary openings 2024″. For example, the flow distributor 2020″ may be formed from a grill, mesh, screen, etc. that is cut to define the target opening 2022″, while the openings/interstices in the grill, mesh, screen, etc. serve as the secondary openings 2024″ which surround the target opening 2022″.

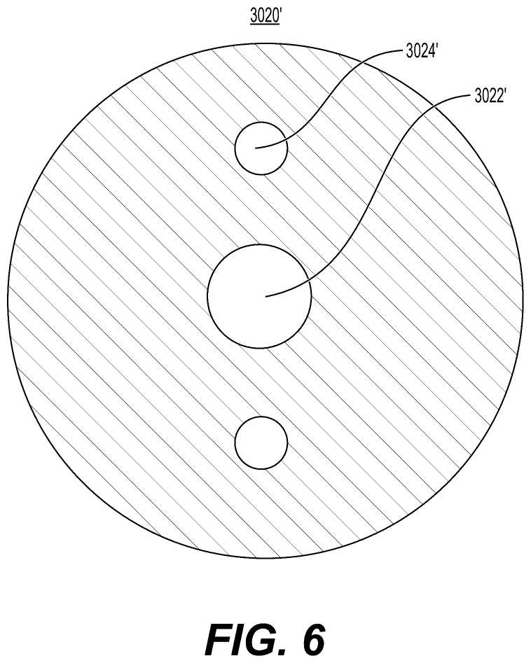

FIG. 5 is a cross-sectional view of another aerosol-generating device 3000 according to an example embodiment. As shown in FIG. 5, the aerosol-generating device 3000 includes a device body 3025, which should be understood to be a schematic depiction of just one of various possible configurations. The device body 3025 is configured to receive an incoming airflow 3010, and a flow distributor 3020 is configured to split the incoming airflow 3010. While the flow distributor 2020 illustrated in FIG. 2 included two holes above the aerosol-forming article 200 (e.g., leading to a bypass channel above the aerosol-forming article 200) and two holes below the aerosol-forming article 200 (e.g. leading to a bypass channel below the aerosol-forming article 200), the flow distributor 3020 illustrated in FIG. 5 includes a single hole above the aerosol-forming article 300 and a single hole below the aerosol-forming article 300. Differences between the flow distributor 2020 of FIG. 2 and the flow distributor 3020 of FIG. 5 will be described further below with reference to FIG. 3 and FIG. 6.

The aerosol-forming article 300 includes an aerosol-forming substrate 360, and a heater 340. The analogous features of the aerosol-forming article 300 in FIG. 5 may be as described in connection with the aerosol-forming article 200 in FIG. 2. Thus, the details of the analogous features of the aerosol-forming article 300 may not be repeated herein in the interest of brevity. The heater 340 may be operated to heat at least a portion of the aerosol-forming substrate 360 as a target airflow flows through the aerosol-forming substrate 360. For example, the flow distributor 3020 may be configured to split the incoming airflow 3010 into a target airflow that flows through the aerosol-forming substrate 360 (e.g., to generate a heated outflow 3042), and one or more secondary airflows 3044 that bypass the aerosol-forming substrate 360.

The device body 3025 includes another flow distributor 3030 that includes one or more holes for allowing the heated outflow 3042 to escape/exit the aerosol-forming substrate 360, and the secondary airflow 3044 to escape/exit the bypass channels around the aerosol-forming substrate 360. The heated outflow 3042 may then mix with the secondary airflow 3044 to form an aerosol 3050.

FIG. 6 is a plan view of another flow distributor 3020′ according to an example embodiment. As shown in FIG. 6, the flow distributor 3020′ includes a target opening 3022′, and two secondary openings 3024′. The target opening 3022′ may be aligned with, for example, the aerosol-forming substrate 360 illustrated in FIG. 5. The secondary openings 3024′ may be aligned with, for example, the bypass air channels in the device body 3025 of FIG. 5.

Compared with the flow distributor 2020′ of FIG. 3, the flow distributor 3020′ of FIG. 6 includes a smaller target opening 3022′, larger secondary openings 3024′, and only two secondary openings 3024′ (in contrast to the eight secondary openings 2024′ illustrated in FIG. 3). Also, the flow distributor 3020′ of FIG. 6 only includes secondary openings 3024′ above and below the target opening 3022′, without any secondary openings at the sides of the target opening 3022′. As mentioned above, in other example embodiments there may be more or less target openings and secondary openings, smaller or larger target openings and secondary openings, different shapes of target openings and secondary openings, different positions of target openings and secondary openings, etc.

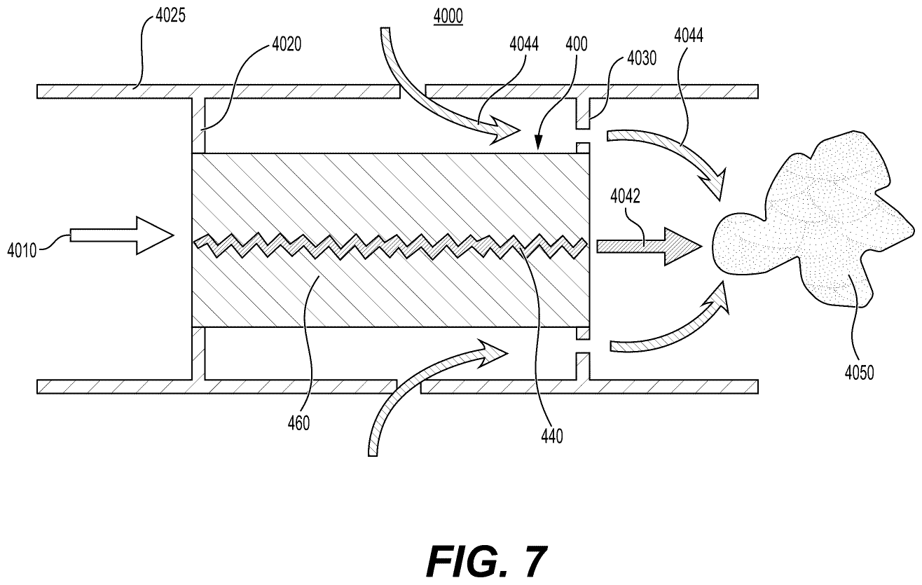

FIG. 7 is a cross-sectional view of another aerosol-generating device 4000 according to an example embodiment. As shown in FIG. 7, the aerosol-generating device 4000 includes a device body 4025, which should be understood to be a schematic depiction of just one of various possible configurations. The device body 4025 is configured to receive an incoming airflow 4010. The device body 4025 also includes a flow distributor 4020.

As shown in FIG. 7, the flow distributor 4020 may not include any openings that allow the incoming airflow 4010 to enter a bypass channel around the aerosol-forming article 400. For example, the flow distributor 4020 may include only one central or axial target opening that allows the incoming airflow 4010 to enter the aerosol-forming article 400. The aerosol-forming article 400 includes an aerosol-forming substrate 460 and a heater 440. The analogous features of the aerosol-forming article 400 in FIG. 7 may be as described in connection with the aerosol-forming article 200 in FIG. 2 and/or the aerosol-forming article 300 in FIG. 5. Thus, the details of the analogous features of the aerosol-forming article 400 may not be repeated herein in the interest of brevity.

In order to allow airflow into the bypass channels around the aerosol-forming substrate 460, the device body 4025 defines holes in the sides of the device body 4025, which allow air to flow into the bypass channel as a secondary airflow 4044. For example, instead of, or in addition to, holes in the flow distributor 4020, the secondary airflow 4044 may enter bypass channels via holes in the side(s) of the device body 4025. In the example embodiment of FIG. 7, the flow distributor 4020 does not include any holes allowing air to enter the bypass channels, so all secondary airflow 4044 enters through holes in the sides of the device body 4025.

The device body 4025 also includes a flow distributor 4030, which allows the heated outflow 4042 to exit the aerosol-forming article 400, and mix with the secondary airflow 4044 after it exits the bypass channels. The secondary airflow 4044 mixes with the heated outflow 4042 to provide an aerosol 4050.

In some example embodiments, similar performance of an aerosol-generating device may be achieved by using a flow distributor to split an incoming airflow into primary and secondary airflows, using separate air inlet holes on sides of the device to allow entry of the secondary airflow, or a combination of both approaches. For example, if 55 cubic centimeters (cc) of air enters the device, 20% (e.g., 11 cc) may be directly drawn into the aerosol-forming substrate, while the remaining 44 cc enters through holes in the sides of the device body, such as holes adjacent a location where the 11 cc aerosol containing air exits the aerosol-forming substrate as a heated outflow. This effect may be the same as or similar to having 55 cc enter an end of the device as an incoming airflow that is split into two by a flow distributor (e.g., 11 cc passes through the target opening of the flow distributor into the aerosol-forming substrate, and 44 cc passes through other holes (e.g., secondary/side openings) in the flow distributor to enter one or more bypass channels). The secondary airflow 4044 illustrated in FIG. 7 may also be referred to, in some instances, as a separate mid-stream secondary airflow. Therefore, in some example embodiments, air may enter the aerosol-generating device through more than one inlet.

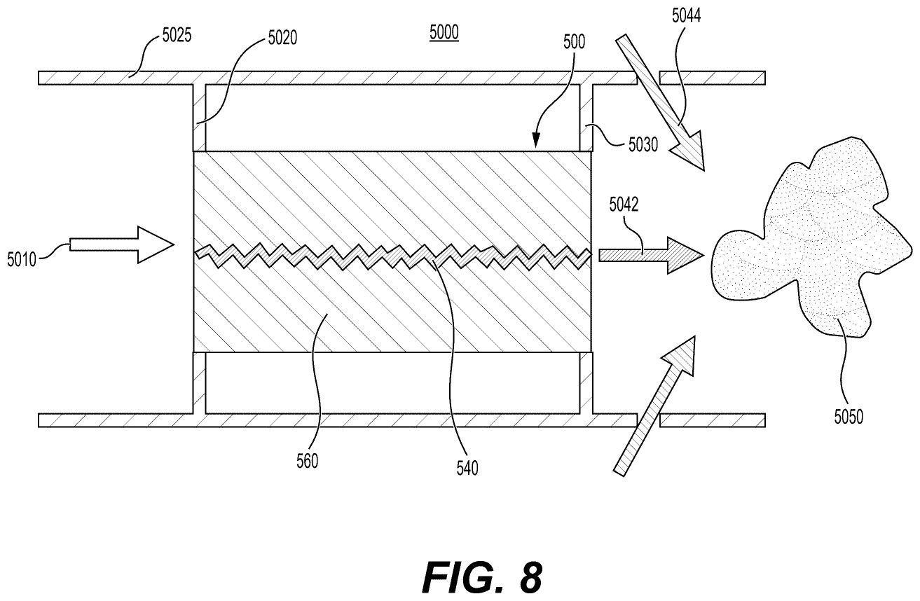

FIG. 8 is a cross-sectional view of another aerosol-generating device 5000 according to an example embodiment. The aerosol-generating device 5000 includes a device body 5025, which may receive an incoming airflow 5010. The illustration of the device body 5025 should be understood to be a schematic depiction of just one of various possible configurations. The aerosol-generating device 5000 also includes a flow distributor 5020. As shown in FIG. 8, the flow distributor 5020 includes only a central target opening that directs all of the incoming airflow 5010 into the aerosol-forming article 500.

The aerosol-forming article 500 includes an aerosol-forming substrate 560, which may be heated by a heater 540. The analogous features of the aerosol-forming article 500 in FIG. 8 may be as described in connection with the aerosol-forming article 200 in FIG. 2, the aerosol-forming article 300 in FIG. 5, and/or the aerosol-forming article 400 in FIG. 7. Thus, the details of the analogous features of the aerosol-forming article 500 may not be repeated herein in the interest of brevity. During the operation of the aerosol-generating device 5000, the incoming airflow 5010 is heated by the heater 540 as it entrains volatiles released from the aerosol-forming substrate 560, and exits the aerosol-forming article 500 through another flow distributor 5030, as a heated outflow 5042. For example, the flow distributor 5030 may include only a central target opening that allows the heated outflow 5042 to exit the aerosol-forming article 500.

As shown in FIG. 8, the secondary airflow 5044 does not travel through the bypass channels above and below the aerosol-forming article 500. For example, the device body 5025 may not include any bypass channels in some instances. Instead, the secondary airflow 5044 enters the device body 5025 through holes in sides of the device body 5025, downstream of the aerosol-forming article 500 and the flow distributor 5030. The secondary airflow 5044 mixes with the heated outflow 5042 to form an aerosol 5050.

Although FIG. 8 illustrates two holes for the incoming secondary airflow 5044, located only downstream of the aerosol-forming article 500 and the flow distributor 5030, other embodiments may include holes downstream of the aerosol-forming article 500 and the flow distributor 5030 in combination with, or in addition to, holes in the sides of the device body 5025 that allow secondary airflow to enter bypass channels, and/or holes in the flow distributor 5020 that split at least a portion of the incoming airflow 5010 to flow through the bypass channels. The aerosol-generating device 5000 may be considered to operate with a separate downstream secondary airflow.

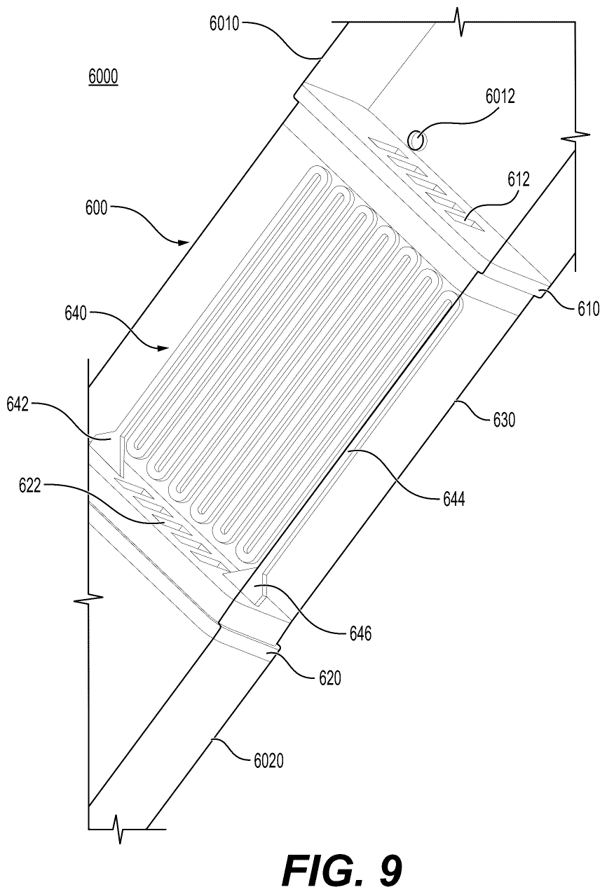

FIG. 9 is a perspective view of another aerosol-generating device 6000 according to an example embodiment. As shown in FIG. 9, the aerosol-generating device 6000 includes a first conduit 6010, a second conduit 6020, and an opening 6012. In some example embodiments, an incoming airflow is received in the second conduit 6020, which passes through the second opening 622 in the second end cap 620, to enter the aerosol-forming article 600. The illustration of the aerosol-generating device 6000 in FIG. 9 should be understood to be a simplified view intended to focus on and facilitate an understanding of the primary and secondary airflows therein. Thus, other structures/components/features of the aerosol-generating device 6000 (e.g., outer housing, mouthpiece, power source, etc.) have not been illustrated to avoid complicating the view.

The aerosol-forming article 600 includes a cover 630 housing a heater 640, and an aerosol-forming substrate (not shown in FIG. 9 to illustrate heater details). The heater 640 includes a first end section 642, an intermediate section 644, and a second end section 646. The heater 640 heats the aerosol-forming substrate as a primary or target airflow enters the aerosol-forming article 600 via the second opening 622.

A heated outflow then exits the aerosol-forming article 600 via the first opening 612 in the first end cap 610, to enter the first conduit 6010. The first conduit 6010 includes an opening 6012, which allows secondary air to enter the first conduit 6010 to mix with the heated outflow from the first opening 612 (e.g., air that passed through the heated aerosol-forming substrate). The opening 6012 in the first conduit is a downstream orifice that allows ambient air to enter the first conduit 6010 to mix with the heated outflow, to provide a mixture of the heated outflow and the secondary air as an aerosol at an outlet of the first conduit 6010.

Although FIG. 9 illustrates one opening 6012 in the first conduit 6010, other embodiments may include more than one opening to allow ambient air to enter the aerosol-generating device 6000, larger or smaller openings, different shapes of openings, openings located at other positions on the first conduit 6010, the second conduit 6020 and/or the cover 630 of the aerosol-forming article 600, etc. Relative amounts of the heated airflow through the aerosol-forming article 600, and the secondary airflow entering the opening 6012, may depend on various factors such as a puff profile, orifice size, tobacco packing density and geometry, etc.

The first end cap 610 and the second end cap 620 may allow for easy connection and disconnection of the first conduit 6010, the second conduit 6020, and the cover 630 of the aerosol-forming article 600. This may facilitate replacement of the aerosol-forming substrate by opening the aerosol-generating device 6000 to refill the aerosol-forming article 600, replacing the aerosol-forming article 600 with a new, prefilled article, etc.

In some example embodiments, the aerosol-forming substrate may include an embedded heater, such as the heater 640 of FIG. 9. The device body may be configured such that the target airflow travels longitudinally through the aerosol-forming substrate and along the embedded heater. The device body may include a power source configured to supply an electric current to the embedded heater to heat the aerosol-forming substrate via resistive heating.

In an example embodiment, the heater 640 is configured to undergo Joule heating (which is also known as ohmic/resistive heating) upon the application of an electric current thereto. Stated in more detail, the heater 640 may be formed of one or more conductors and configured to produce heat when an electric current passes therethrough. The electric current may be supplied to the first end section 642 and the second end section 646 of the heater 640 from a power source (e.g., battery) within the aerosol-generating device 6000. Suitable conductors for the heater 640 include an iron-based alloy (e.g., stainless steel) and/or a nickel-based alloy (e.g., nichrome). The intermediate section 644 of the heater 640 may have a thickness of about 0.1-0.3 mm (e.g., 0.15-0.25 mm) and a resistance of about 0.4-0.8 Ohms (e.g., 0.5-0.7 Ohms).

The electric current from the power source within the aerosol-generating device 6000 may be transmitted via electrodes configured to electrically contact the first end section 642 and the second end section 646 of the heater 640 when the aerosol-forming article 600 is inserted into the aerosol-generating device 6000. In a non-limiting embodiment, the electrodes may be spring-loaded to enhance an engagement with the heater 640 of the aerosol-forming article 600. Also, the movement (e.g., engagement, release) of the electrodes may be achieved by mechanical actuation. Furthermore, the supply of the electric current from the aerosol-generating device 6000 to the aerosol-forming article 600 may be a manual operation (e.g., button-activated) or an automatic operation (e.g., puff-activated). Additional details of the aerosol-forming article 600 and the aerosol-generating device 6000 may be as described in connection with the capsule 1200 and the aerosol-generating device 100/500, respectively, in U.S. application Ser. No. 17/981,973, filed Nov. 7, 2022, titled “CAPSULES HAVING ELECTRICAL CONTACT PADS WITH SURFACE DISCONTINUITIES AND HEAT-NOT-BURN (HNB) AEROSOL-GENERATING DEVICES INCLUDING THE SAME,” Atty. Dkt. No. 24000NV-000874-US, the disclosure of which is incorporated herein in its entirety by reference.

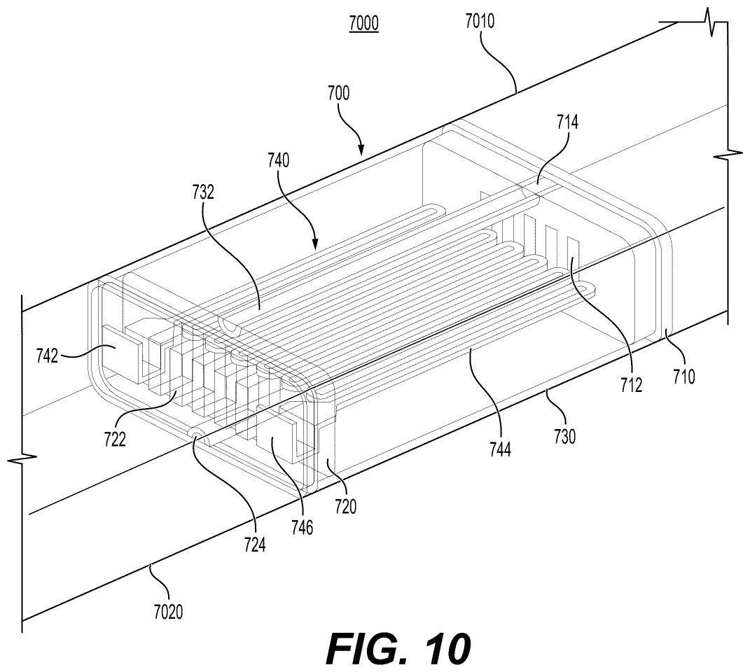

FIG. 10 is a perspective view of another aerosol-generating device 7000 according to an example embodiment. The aerosol-generating device 7000 includes a first conduit 7010 and a second conduit 7020. Between the first conduit 7010 and the second conduit 7020 is an aerosol-forming article 700 including a cover 730, a first end cap 710, and a second end cap 720. The first end cap 710 is removably coupled with the first conduit 7010 (e.g., via a friction fit, etc.), and the second end cap 720 is removably coupled with the second conduit 7020.

The aerosol-forming article 700 includes a heater 740 and an aerosol-forming substrate (not shown in FIG. 10 for purposes of illustrating heater details). The heater 740 includes a first end section 742, an intermediate section 744, and a second end section 746. The heater 740 is configured to heat the aerosol-forming substrate as a target or primary airflow moves through the aerosol-forming substrate and over, along, through, or otherwise in thermal communication with the heater 740.

For example, the second conduit 7020 receives an incoming airflow at an end (e.g., upstream end) of the second conduit 7020 opposite the aerosol-forming article 700. At least a portion of the incoming airflow passes through a second opening 722 in the second end cap 720, to enter the aerosol-forming article 700. This portion of the split incoming airflow may be referred to as a target or primary airflow.

The aerosol-forming article 700 also includes a channel 732 including a first aperture 714 at a first end of the channel 732, and a second aperture 724 at a second end of the channel 732 opposite the first end. As shown in FIG. 10, the first aperture 714 is an opening or orifice in the first end cap 710, and the second aperture 724 is an opening or orifice defined in the second end cap 720.

The channel 732 may be considered as a bypass channel, which allows a secondary airflow to pass through the aerosol-forming article 700 without coming into contact with the heater 740 or the aerosol-forming substrate (not shown). For example, the channel 732 may provide a barrier that inhibits or prevents the secondary airflow from thermally interacting with the heater 740, or traveling through the aerosol-forming substrate. Therefore, the secondary airflow in the channel 732 does not absorb as much (if any) of the heat generated by the heater 740 as the heated airflow that passes over the heater 740 and through the aerosol-forming substrate.

The second end cap 720 may operate as a flow distributor, which splits the incoming airflow received at the second conduit 7020. For example, the incoming airflow may be split by the second end cap 720 into a target or primary airflow that passes through the second opening 722 in the second end cap 720 to be heated by the heater 740, and a separate secondary airflow that passes through the second aperture 724 to enter one or more channels 732 to bypass the heater 740 and the aerosol-forming substrate.

The first end cap 710 allows the heated outflow exiting via the first opening 712 to mix with the secondary airflow exiting the channels 732 via the first apertures 714. Although FIG. 10 illustrates two channels 732 above and below the heater 740, other example embodiments may include more or less channels, channels having different shapes, channels positioned in other locations of the aerosol-forming article 700, etc. Similarly, the first end cap 710 and the second end cap 720 may include more or less apertures, apertures having different shapes, apertures in different locations on the end caps, etc.

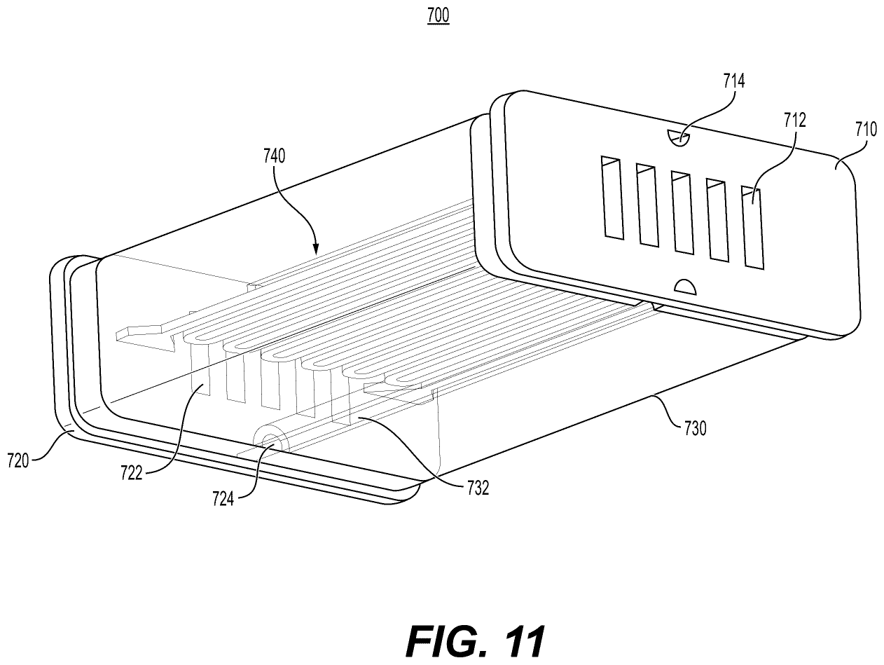

FIG. 11 is a downstream, perspective view of the aerosol-forming article 700 in FIG. 10. As mentioned above, the aerosol-forming article 700 includes a first end cap 710 including a first opening 712 and two first apertures 714. The aerosol-forming article 700 also includes a second end cap 720 including a second opening 722 and two second apertures 724.

Between the first end cap 710 and the second end cap 720 are two channels 732 above and below the heater 740, which may allow the secondary airflow received at the second aperture 724 to bypass the heater 740. A cover 730 is also positioned between the first end cap 710 and the second end cap 720, to house an aerosol-forming substrate (not shown in FIG. 11).

Although FIG. 11 illustrates the first opening 712 and the second opening 722 as each including five slots, other example embodiments may include more or less openings, openings having different shapes, openings in other locations, etc. Also, although example embodiments have been described herein with airflow passing from the second end cap 720 to the first end cap 710, in other example embodiments the airflow may occur in the opposite direction (e.g., from the first end cap 710 towards the second end cap 720).

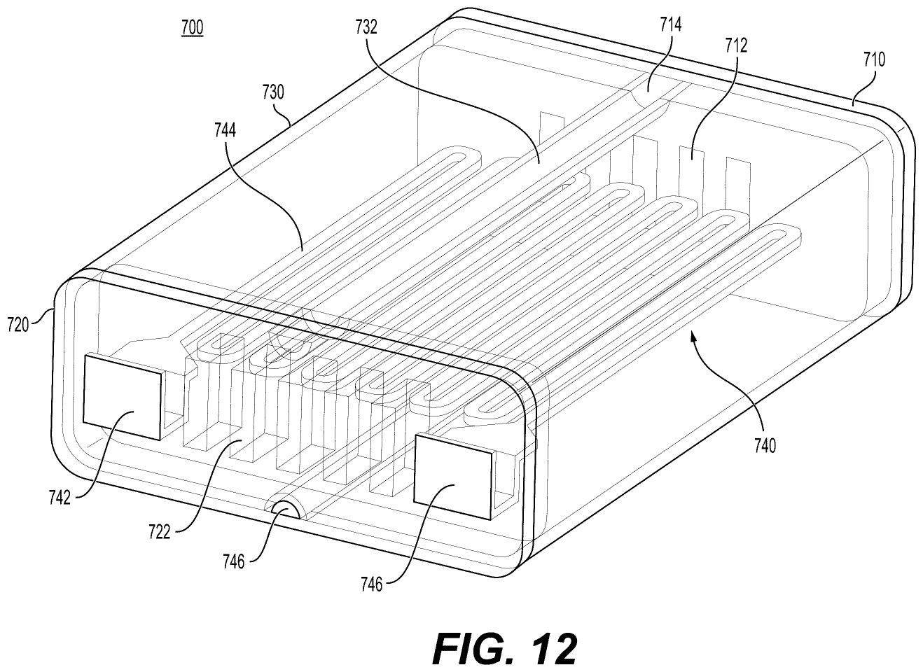

FIG. 12 is an upstream, perspective view of the aerosol-forming article 700 in FIG. 10. FIG. 12 illustrates two channels 732, above and below the heater 740. The heater 740 includes a first end section 742, a second end section 746, and an intermediate section 744 between the first end section 742 and the second end section 746. The intermediate section 744 includes approximately seven S-bends arranged parallel to one another in a single plane, but other embodiments may include heaters having different shapes, heaters that are non-planar, heaters having more or less looped sections, etc.

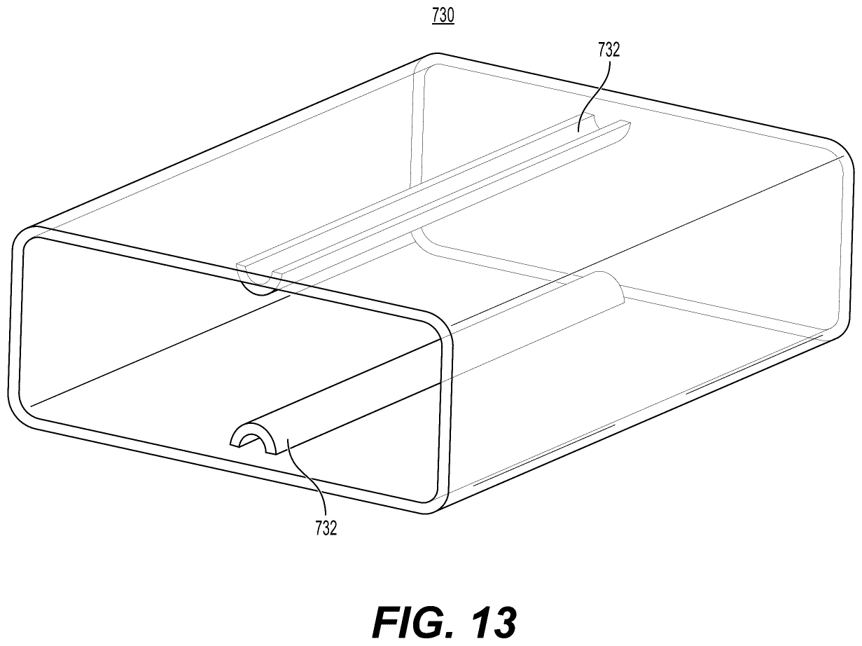

FIG. 13 is a perspective view of the cover 730 of the aerosol-forming article 700 in FIG. 10. As shown in FIG. 13, the cover 730 includes two channels 732, both centered on opposite sides of the cover 730. The channels 732 may be integral with the cover 730, and allow secondary airflow to bypass a heater and aerosol-forming substrate positioned within the cover 730 when the aerosol-forming article is assembled. Although FIG. 13 illustrates two channels 732 having a semicircular cross-sectional profile, other covers 730 may include more or less channels, channels having other shapes (e.g., cross-sectional profiles), channels in other locations, etc. In some example embodiments, one or more bypass channels may not be integral with the cover 730, may not contact the cover 730, etc.

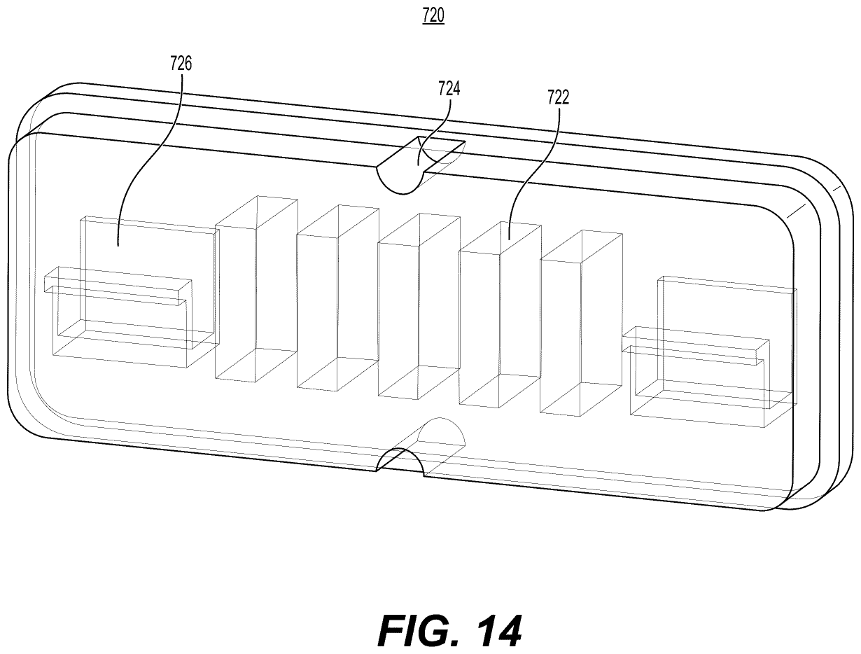

FIG. 14 is a perspective view of the second end cap 720 of the aerosol-forming article 700 in FIG. 10. As shown in FIG. 14, the second end cap 720 includes a second opening 722, and two second apertures 724. The second end cap 720 may split an incoming airflow to allow a portion of the incoming airflow to pass through the openings 722 as a target or primary airflow that will be warmed by the heater, and another portion of the incoming airflow that will pass through the second apertures 724 to bypass the heater via one or more bypass channels. Although FIG. 14 illustrates two second apertures 724 and five slots for the openings 722, other example embodiments may include more or less apertures, more or less openings, apertures and/or openings having different shapes, apertures and/or openings in other locations on the second end cap 720, etc.

The second end cap 720 also includes two second orifices 726. The second orifices 726 may allow the first end section 742 and the second end section 746 of the heater 740 to pass through the second end cap 720. For example, the first end section 742 and the second end section 746 may pass through the second orifices 726 to electrically contact a power source such as a battery, in order to receive current for heating the intermediate section 744 of the heater 740.

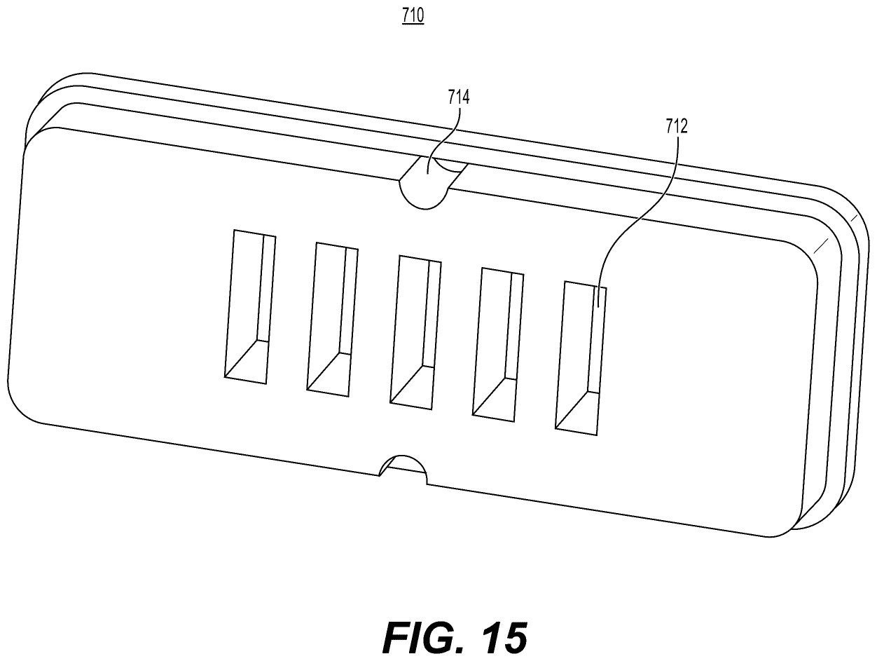

FIG. 15 is a perspective view of the first end cap 710 of the aerosol-forming article 700 in FIG. 10. As shown in FIG. 15, the first end cap 710 includes a first opening 712, and two first apertures 714. The first end cap 710 may allow a heated outflow exiting the aerosol-forming article 700 through the first opening 712 to mix with secondary air exiting the first apertures 714. Although FIG. 15 illustrates two first apertures 714 and five slots for the first openings 712, other example embodiments may include more or less apertures, more or less openings, apertures and/or openings having different shapes, apertures and/or openings in other locations on the first end cap 710, etc.

FIG. 16 is a thermal imaging view of an aerosol-forming article wherein all of the incoming airflow is passing through the aerosol-forming substrate. As shown in FIG. 16, when 100% of the incoming airflow passes through the aerosol-forming substrate and over the heater, only a small portion 1602 near the center of the heater reaches a desired heating temperature (as shown by the dark circle in the center of the heater). For example, only the dark center circle portion 1602 of the heater in FIG. 16 may reach a temperature of, e.g., approximately 290 degrees Celsius, while other peripheral portions of the heater remain at lower temperatures due to the 100% incoming airflow over the heater.

FIG. 17 is a thermal imaging view of an aerosol-forming article wherein only a fraction of the incoming airflow is passing through the aerosol-forming substrate. For example, in FIG. 17 a flow distributor upstream of the heater may divert 80% of the incoming airflow to a secondary airflow through one or more bypass channels, such that only 20% of the incoming airflow passes through the aerosol-forming substrate and over the heater as a primary or target airflow. Compared with the 100% airflow over the heater example illustrated in FIG. 16, the 20% incoming airflow over the heater example in FIG. 17 results in a greater area of the heater reaching higher temperatures. For example, the larger area 1702 of darker color on the heater in FIG. 17 illustrates that a greater portion of the heater reaches a desired operating temperature, e.g., approximately 290 degrees Celsius, when only a portion of the split incoming airflow passes over the heater.

Table 1 below illustrates an example of an amount of aerosol-forming substrate (e.g., tobacco) that is exposed to four temperature thresholds, for four example cases. The first example case is when the incoming airflow is not split at all, such as the sample temperature profile in FIG. 16. The second example case is where the incoming airflow is split such that 50% passes over the heater and 50% bypasses the heater as secondary airflow, the third example case illustrates only 20% of the incoming air flowing over the heater (as illustrated in the example temperature profile of FIG. 17), and the fourth example case illustrates only 10% of the incoming airflow passing over the heater. As shown in the table, reducing the amount of incoming airflow that actually passes over the heater increases the amount of aerosol forming substrate that is exposed to higher heater temperature. In various implementations, the heating may result in at least 70% of the aerosol-forming substrate having a temperature of 150° C. or higher.

TABLE 1
% Tobacco Exposed

| Temp (deg C.) | No split | 50/50 split | 20/80 split | 10/90 split |
|---|---|---|---|---|
| >290 | 0.1% | 0.1% | 0.2% | 0.3% |
| >250 | 4.6% | 5.9% | 8.1% | 8.6% |
| >200 | 27.0% | 30.2% | 33.4% | 34.1% |
| >150 | 69.2% | 74.8% | 83.4% | 86.5% |

As described above, the device body of an aerosol-generating device may be configured such that the target airflow directed through the aerosol-forming substrate is a fraction of the incoming airflow drawn into the device body. For example, the device body may be configured to split the incoming airflow into the target airflow and the secondary airflow, and the device body may be configured such that the secondary airflow bypasses the aerosol-forming substrate.

In some example embodiments, the target airflow may be less than the secondary airflow. For instance, the target airflow may be 50% or less of the incoming airflow while the secondary airflow may be 50% or more of the incoming airflow. In another instance, the target airflow may be 20% or less of the incoming airflow while the secondary airflow may be 80% or more of the incoming airflow. In a further instance, the target airflow may be 10% or less of the incoming airflow while the secondary airflow may be 90% or more of the incoming airflow.

In some example embodiments, the device body may include a flow distributor configured to split the incoming airflow. The flow distributor may be disposed upstream from the aerosol-forming substrate when the aerosol-forming substrate is received within the device body. The flow distributor may be in a form of a baffle defining a plurality of holes. One of the plurality of holes may be configured to split the incoming airflow into the target airflow that is directed through the aerosol-forming substrate, while a remainder of the plurality of holes may be configured to split/direct the incoming airflow into the secondary flow so as to bypass the aerosol-forming substrate.

The device body may be configured such that the target airflow directed through the aerosol-forming substrate is substantially all of the incoming airflow drawn into the device body, and the secondary airflow may be an additional stream drawn into the device body at a location different from incoming airflow. In some example embodiments, the aerosol-generating device may further comprise an aerosol-forming article (such as a capsule, container, etc.), configured to contain the aerosol-forming substrate. The capsule may define at least one channel configured to direct the secondary airflow so as to bypass the aerosol-forming substrate. The aerosol-forming article may be configured to be received within an aerosol-generating device (e.g., heat-not-burn aerosol-generating device).

Although some example aerosol-forming articles are shown in the figures as having a rectangular shape, it should be understood that other configurations may be employed. For instance, the shape may be circular such that the aerosol-forming article has a disk-like appearance. In another instance, the shape of the aerosol-forming article may be elliptical or racetrack-like. In other instances, the aerosol-forming article may have a polygonal shape (regular or irregular), including a triangle, a square, a pentagon, a hexagon, a heptagon, or an octagon. The laminar structure and generally planar form of the aerosol-forming article may facilitate stacking so as to allow a plurality of aerosol-forming articles to be stored in an aerosol-generating device or other receptacle for dispensing a new aerosol-forming article or receiving a depleted aerosol-forming article.

The aerosol-forming article may include a housing and a heater within the housing. The housing of the aerosol-forming article has interior surfaces defining a chamber configured to hold an aerosol-forming substrate. In addition, the housing of the aerosol-forming article may be viewed as having exterior surfaces constituting a first face, an opposing second face, and a side face of the aerosol-forming article. The first face (e.g., first end face) and the second face (e.g., second end face) of the aerosol-forming article may be permeable to an aerosol. The side face of the aerosol-forming article is between the first face and the second face. The side face may be regarded as a periphery of the aerosol-forming article.

Referring again to FIG. 1, the aerosol-generating device 1000 (e.g., heat-not-burn aerosol-generating device) may include a mouthpiece on the device body 1025. A power source 1035 and control circuitry 1045 may be disposed within the device body 1025 of the aerosol-generating device 1000. The power source 1035 may include one or more batteries (e.g., rechargeable dual battery arrangement). The aerosol-generating device 1000 may be configured to receive the aerosol-forming article 100, which may be as described in connection with any of the embodiments herein. The aerosol-generating device 1000 may include an engagement assembly configured to electrically contact the aerosol-forming article 100. In an example embodiment, the engagement assembly includes a first electrode 1055a and a second electrode 1055b configured to electrically contact a first end section and a second end section, respectively, of a heater of the aerosol-forming article 100.

When the aerosol-forming article 100 is inserted into the aerosol-generating device 1000, the control circuitry 1045 may instruct the power source 1035 to supply an electric current to the first electrode 1055a and the second electrode 1055b of the engagement assembly. The supply of current from the power source 1035 may be in response to a manual operation (e.g., button-activation) or an automatic operation (e.g., puff-activation). As a result of the current, the aerosol-forming article 100 may be heated to generate an aerosol. The aerosol generated may be drawn from the aerosol-generating device 1000 via an aerosol outlet 1015 in the mouthpiece.

Further to the non-limiting embodiments set forth herein, additional details of the substrates, capsules, devices, and methods discussed herein may also be found in U.S. application Ser. No. 16/451,662, filed Jun. 25, 2019, titled “CAPSULES, HEAT-NOT-BURN (HNB) AEROSOL-GENERATING DEVICES, AND METHODS OF GENERATING AN AEROSOL,” Atty. Dkt. No. 24000NV-000522-US; U.S. application Ser. No. 16/252,951, filed Jan. 21, 2019, titled “CAPSULES, HEAT-NOT-BURN (HNB) AEROSOL-GENERATING DEVICES, AND METHODS OF GENERATING AN AEROSOL,” Atty. Dkt. No. 24000NV-000521-US; U.S. application Ser. No. 15/845,501, filed Dec. 18, 2017, titled “VAPORIZING DEVICES AND METHODS FOR DELIVERING A COMPOUND USING THE SAME,” Atty. Dkt. No. 24000DM-000012-US; and U.S. application Ser. No. 15/559,308, filed Sep. 18, 2017, titled “VAPORIZER FOR VAPORIZING AN ACTIVE INGREDIENT,” Atty. Dkt. No. 24000DM-000003-US-NP, the disclosures of each of which are incorporated herein in their entirety by reference.

While a number of example embodiments have been disclosed herein, it should be understood that other variations may be possible. Such variations are not to be regarded as a departure from the spirit and scope of the present disclosure, and all such modifications as would be obvious to one skilled in the art are intended to be included within the scope of the following claims.

1. An aerosol-generating device, comprising:
an aerosol-forming substrate; and
a device body configured to receive the aerosol-forming substrate and an incoming airflow, wherein the device body is configured to direct a target airflow of the incoming airflow through the aerosol-forming substrate while the aerosol-forming substrate is heated such that a heated outflow exits therefrom, the device body is configured to combine the heated outflow with a secondary airflow to produce a mixed flow, and the secondary airflow is an air stream that has not passed through the aerosol-forming substrate.

2. The aerosol-generating device of claim 1, wherein the aerosol-forming substrate includes a plant material.

3. The aerosol-generating device of claim 2, wherein the plant material includes tobacco.

4. The aerosol-generating device of claim 1, wherein the aerosol-forming substrate includes an embedded heater.

5. The aerosol-generating device of claim 4, wherein the device body is configured such that the target airflow travels longitudinally through the aerosol-forming substrate and along the embedded heater.

6. The aerosol-generating device of claim 4, wherein the device body includes a power source configured to supply an electric current to the embedded heater to heat the aerosol-forming substrate via resistive heating.

7. The aerosol-generating device of claim 1, wherein the device body is configured such that the target airflow directed through the aerosol-forming substrate is a fraction of the incoming airflow drawn into the device body.

8. The aerosol-generating device of claim 1, wherein the device body is configured to split the incoming airflow into the target airflow and the secondary airflow.

9. The aerosol-generating device of claim 1, wherein the device body is configured such that the secondary airflow bypasses the aerosol-forming substrate.

10. The aerosol-generating device of claim 1, wherein the target airflow is less than the secondary airflow.

11. The aerosol-generating device of claim 10, wherein the target airflow is 20% or less of the incoming airflow while the secondary airflow is 80% or more of the incoming airflow.

12. The aerosol-generating device of claim 11, wherein the target airflow is 10% or less of the incoming airflow while the secondary airflow is 90% or more of the incoming airflow.

13. The aerosol-generating device of claim 1, wherein the device body includes a flow distributor configured to split the incoming airflow, the flow distributor disposed upstream from the aerosol-forming substrate when the aerosol-forming substrate is received within the device body.

14. The aerosol-generating device of claim 13, wherein the flow distributor comprises a baffle defining a plurality of holes.

15. The aerosol-generating device of claim 14, wherein one of the plurality of holes is configured to split the incoming airflow into the target airflow that is directed through the aerosol-forming substrate, while a remainder of the plurality of holes is configured to split the incoming airflow into the secondary airflow so as to bypass the aerosol-forming substrate.

16. The aerosol-generating device of claim 1, wherein the device body is configured such that the target airflow directed through the aerosol-forming substrate is substantially all of the incoming airflow drawn into the device body, and the secondary airflow is an additional stream drawn into the device body at a location different from incoming airflow.

17. The aerosol-generating device of claim 1, further comprising:
a capsule configured to contain the aerosol-forming substrate.

18. The aerosol-generating device of claim 17, wherein the capsule defines at least one channel configured to direct the secondary airflow so as to bypass the aerosol-forming substrate.

19. A capsule for an aerosol-generating device, comprising:
a housing defining a substrate chamber, a chamber inlet, a chamber outlet, a bypass channel, a channel inlet, and a channel outlet; and
an aerosol-forming substrate within the substrate chamber of the housing, wherein the housing is configured to split an incoming airflow into a target airflow and a secondary airflow such that the target airflow is directed through the aerosol-forming substrate via the chamber inlet, the substrate chamber, and the chamber outlet and such that the secondary airflow bypasses the aerosol-forming substrate via the channel inlet, the bypass channel, and the channel outlet.

20. A method of generating an aerosol, comprising:
heating an aerosol-forming substrate; and
combining a heated outflow from the aerosol-forming substrate with a secondary airflow to produce a mixed flow, wherein the secondary airflow is an air stream that has not passed through the aerosol-forming substrate.
